(19) United States
(12) Patent Application Publication
(10) Pub. No.: US 2025/0001408 A1
(43) Pub. Date: Jan. 2, 2025

(54) INTEGRATED LATERAL FLOW BIOASSAY AND BIOSENSOR

(71) Applicant: MX3 Diagnostics, Inc., Austin, TX (US)

(72) Inventors: Chathurika Darshani Abeyrathne, Mitcham (AU); Michael Erlichster, Caulfield North (AU); Gursharan Chana, Fitroy North (AU); Efstratios Skafidas, Thornbury (AU)

(21) Appl. No.: 18/688,580

(22) PCT Filed: Sep. 1, 2022
(86) PCT No.: PCT/US2022/075863

§ 371 Date: Mar. 1, 2024

Related U.S. Application Data
(60) Provisional application No. 63/240,732, filed on Sep. 3, 2021.

(51) Int. Cl. B01L 3/00

(52) U.S. Cl. CPC B01L 3/5023; B01L 2200/143; B01L 2300/023; B01L 2300/0645; B01L 2300/0663; B01L 2300/0825; B01L 2300/1827

(57) ABSTRACT

Method and systems for quantifying analytes in biological samples include a lateral flow assay (LFA). Integrated LFA analyte measuring devices include a first sensor that generates a signal relative to the concentration of a measured analyte. The LFA device may also include one or multiple additional sensors for auxiliary measures to assist in correcting, adjusting, or interpreting the output of the first sensor. The LFA device can cooperate with a remote database for data storage, analysis, and access.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### TECHNICAL FIELD

This application describes biochemical systems and methods. More specifically, the application describes method and systems for quantifying analytes in biological samples. The method and quantification of analytes will aid in diagnosis, treatment, and management of diseases and/or monitor health and wellbeing, human performance, or to aid in law enforcement.

### BACKGROUND

Techniques for rapid identification and quantification of analytes in biological samples are essential for many applications including diagnosis, treatment and management of diseases, identification of biological threats, maintaining normal health and wellbeing, optimizing human performance, and identifying controlled substances.

Immunoassays are one standard method of analyzing biological samples. The traditional immunoassay approach is the enzyme-linked immunosorbent assay (ELISA). ELISA testing has several drawbacks. It is a complex multiple-step processes involving antibody-antigen complexation, multiple repeated wash steps, manual addition of reagents and extended analysis time with expensive laboratory equipment. Furthermore, significant training in laboratory technique is required to perform laboratory ELISA testing.

An alternative to ELISA testing are biosensors in which a measurable signal is generated in the presence of a recognition molecule. Biosensors based on resonant, optical, thermal, and electrochemical measurements have been developed for point-of-care (POC) testing applications. When implemented appropriately, these approaches simplify testing processes and shorten assay time, while retaining sufficient accuracy, precision, and selectivity for given applications.

While biosensors have been extensively reported in academic literature with proof-of-concept demonstrations, they have not been widely used for in-field or POC diagnostics (with few notable exceptions, such as pregnancy tests, as mentioned below).

The lack of wide adoption of biosensors may be due to the complexity of mass manufacturing of many reported designs. Reported processes may require many steps (immobilization of antigens or antibodies, blocking, washing), relatively expensive materials and clean-room infrastructure. There remains a need for portable, affordable, rapid, sensitive, and specific diagnostic biosensors, particularly in low-middle income countries and other resource-limited areas.

Lateral flow Assays (LFA) have several advantages over other biosensor platforms, including a relative low cost, established manufacturing processes for lateral flow immunochromatographic assays, and excellent shelf life. While qualitative paper based LFA test strips, such as pregnancy test kits, are commonly available in the market, quantitation of biomarker concentration is still a challenge due to ambiguity in chromatographic output. This limits test results to applications where a binary present/absent test result is acceptable. By integrating lateral flow strips with a quantitative element, more informative data may be recorded, enabling novel applications.

Integration of quantitative elements introduces several additional challenges. Firstly, biological samples such as blood, urine, or saliva, may vary dramatically in pH and ionic content or contain contaminants which may influence test performance and impact reliability of test results. One method of overcoming these confounding factors is processing or pre-processing of the sample prior to analysis to normalize ionic content and remove contaminants. However, this pre-processing introduces additional complexity and cost to the testing procedure. Ideally a test system would not require sample processing prior to sample analysis.

Secondly, quantitative elements may require precise timing between sample addition and instigation of a secondary measurement process or the initiation of measurement to allow for a defined incubation period. Ideally a test system would integrate an automatic or intuitive timing element to reduce the potential for user error or error introduced by variation in sample viscosity in the measurement process and improve the accuracy of results.

Thirdly, quantitative methods may require the precise and well-directed addition of reagents following sample incubation. Ideally a test system would integrate automatic or user-friendly elements which direct and control the addition of such reagents.

The novel quantitative lateral flow biosensors described in this patent addresses the above challenges allowing for an integrated, low cost, portable and rapid quantitative testing tool which does not require highly skilled personnel to administer.

### SUMMARY

The assignee of the present application has filed previous patent applications describing systems, methods and devices for testing, measuring and analyzing saliva, to measure a subject's hydration level, as well as for measuring other substances (e.g., sweat) and/or physiological parameters in a human or animal subject. These previous patent applications include U.S. patent application Ser. No. 16/197,530, filed Nov. 21, 2018; Ser. No. 16/598,000, filed Oct. 11, 2018; Ser. No. 17/159,770 filed Jan. 27, 2021; and Ser. No. 17/149,181 filed on Jan. 14, 2021. All of these above-referenced patent applications are hereby incorporated by reference into the present application, and they may be referred to below as “the Incorporated Applications.”

The present application adds to the technologies in the Incorporated Applications by describing a system and method for identification or quantifying analytes in biological samples.

These and other aspects and implementations are described in greater detail below, in relation to the attached drawing figures.

In some aspects, the devices and techniques described herein relate to a system for quantifying the concentration of one or more analytes in a biological sample using a lateral flow system with: a first sensor which generates a signal relative to the concentration of the one or more analytes; one or more additional sensors for one or more respective auxiliary measures to assist in interpretation of the signal of the first sensor; and an analysis device which facilitates testing.

In some aspects, one of the additional sensors is integrated into the lateral flow system. In some aspects, one of the additional sensors uses the same analysis device but is not integrated into the lateral flow system. In some aspects, one of the auxiliary measures is pH. In some aspects, one of the auxiliary measures is the concentration of an ion. In some aspects, one of the auxiliary measures is osmolarity. In some aspects, one of the auxiliary measures is temperature.

In some aspects, one of the auxiliary measures is used to correct the signal of the first sensor. In some aspects, one of the auxiliary measures is used to flag an error. In some aspects, one of the auxiliary measures is used to provide diagnostic context for the signal of the first sensor. In some aspects, one of the auxiliary measures is data collected with a secondary analysis device. In some aspects, one of the auxiliary measures is a set of answers provided by an individual providing the biological sample. In some aspects, one of the auxiliary measures is a set of observations annotated by an individual operating the system. In some aspects, one of the auxiliary measures is used to provide diagnostic context for the signal of the first sensor.

In some aspects, the techniques described herein relate to a system for quantifying the concentration of one or more analytes in a biological sample using a lateral flow system with: one or more sensors which each generate a signal relative to the concentration of a respective one of the one or more analytes; integrated microfluidics and materials for collection and processing of the biological sample; and an analyzer device which facilitates testing.

In some aspects, the integrated microfluidics and materials control for physical variability of the biological sample. In some aspects, the physical variability includes viscosity or bubble content. In some aspects, the integrated microfluidics and materials control for chemical variability of the biological sample. In some aspects, the chemical variability includes pH or ionic content.

In some aspects, the techniques described herein relate to a system for quantifying the concentration of one or more analytes in a biological sample using a lateral flow system with: one or more sensors which each generate a signal relative to the concentration of a respective one of the one or more analytes; one or more integrated automatic timing elements; and an analyzer device which facilitates testing.

In some aspects, the timing elements include multiple membranes of variable flow speed. In some aspects, the timing elements include a fluid detection electrode which initiates a timer on the analyzer device. In some aspects, the timing elements include a fluid detection electrode which initiates a measurement. In some aspects, the timing element includes a fluid detection electrode which is positioned to identify that a sufficient sample has been collected. In some aspects, an output of the timing elements is used to flag an error.

In some aspects, the techniques described herein relate to a system for quantifying the concentration of one or more analytes in a biological sample using a lateral flow system with: one or more sensors which each generates a signal relative to the concentration of a respective one of the one or more analytes; an integrated packaging of liquid required to initiate a chemical reaction; and an analyzer device which facilitates testing.

In some aspects, the integrated packaging of liquid is manually opened by a system operator to initiate a step in a measurement process. In some aspects, the integrated packaging of liquid is automatically opened by the analyzer device to initiate a step in a measurement process.

In some aspects, the techniques described herein relate to a system for quantifying the concentration of one or more analytes in a biological sample using a lateral flow system with: one or more sensors which each generates a signal relative to the concentration of a respective one of the one or more analytes; an integrated cutting element to isolate a segment of the lateral flow system; and an analyzer device which facilitates testing.

In some aspects, the cutting element is combined with an integrated liquid pack.

In some aspects, the techniques described herein relate to a system for quantifying the concentration of one or more analytes in a biological sample using a lateral flow system with: one or more integrated ion selective electrodes; ion loaded liposomes; a conjugate molecule which acts to lyse the liposomes; and an analyzer device which facilitates testing. In some aspects, the ion loaded liposomes are immobilized close to the one or more ion selective electrodes. In some aspects, the ion loaded liposomes are tagged to a capture ligand. In some aspects, the presence of a target analyte in the biological sample prevents capture of the conjugate molecule by a capture ligand, leading to lysis of the liposome and an increase in ion concentration.

In some aspects, the techniques described herein relate to a system for quantifying the concentration of one or more analytes in a biological sample using a lateral flow system with: one or more integrated electrodes; one or more molecularly imprinted polymers targeted at the one or more analytes; one or more labeled conjugate molecules; and an analyzer device which facilitates testing.

In some aspects, each of the one or more molecularly imprinted polymers is positioned on a respective one of the one or more integrated electrodes. In some aspects, at least one of the labeled conjugate molecules compete with at least one of the analytes to bind to at least one of the molecularly imprinted polymers. In some aspects, one of the labeled conjugate molecules binds to one or more analytes and the resulting complex binds to one of the molecularly imprinted polymers.

In some aspects, the techniques described herein relate to a system for quantifying the concentration of one or more analytes in a biological sample using a lateral flow system with: one or more integrated electrodes; one or more aptamers targeted at the one or more analytes; one or more labeled conjugate molecules; and an analyzer device which facilitates testing.

In some aspects, the aptamers are each positioned on a respective one of the integrated electrodes. In some aspects, the labeled conjugate molecules compete with the analytes to bind to an aptamer. In some aspects, the labeled conjugate molecules bind to one or more analytes and the resulting complex binds to an aptamer.

In some aspects, the techniques described herein relate to a system for quantifying the concentration of one or more analytes in a biological sample using a lateral flow system with: one or more integrated electrodes; one or more temperature modifying elements; and an analyzer device which facilitates testing.

In some aspects, the temperature modifying elements include a resistive heating element integrated into the lateral flow system. In some aspects, the temperature modifying elements include a liquid pack which when opened results in an endothermic reaction, lowering the temperature of the lateral flow system. In some aspects, the temperature modifying elements are located within the analyzer device and the temperature of the lateral flow system is modified using a thermally conductive material. In some aspects, the analyzer device prompts a user to only apply a sample for analysis once a desired temperature has been reached.

In some aspects, the techniques described herein relate to a system for quantifying the concentration of one or more analytes in a biological sample using a lateral flow system with: one or more integrated electrodes; one or more integrated quality control elements; and an analyzer device which facilitates testing.

In some aspects, the quality control elements are irreversibly modified by exposure to a temperature outside of a desired range. In some aspects, the quality control elements are irreversibly modified by exposure to a humidity outside of a desired range. In some aspects, the quality control elements are irreversibly modified once the lateral flow system has been used. In some aspects, the analyzer device automatically measures at least one of the one or more quality control elements and does not perform analysis when the at least one quality control element indicates inappropriate storage or use.

In some aspects, the techniques described herein relate to a system for quantifying the concentration of one or more analytes in a biological sample using a lateral flow system with: one or more integrated electrodes for type and/or batch tracking; an analyzer device which facilitates testing; and a mobile application.

In some aspects, the type and/or batch tracking is accomplished using the resistance value of an integrated electrode. In some aspects, the type and/or batch tracking is used to indicate whether a lateral flow test is out of date.

In some aspects, the techniques described herein relate to a system for quantifying the concentration of one or more analytes in a biological sample using a lateral flow system with: one or multiple integrated electrodes; an analyzer device which facilitates testing; and a mobile application.

In some aspects, the analyzer device wirelessly communicates measurement data with an internet connected mobile device. In some aspects, the system further includes a centralized database for receiving measurement data from the analyzer device, wherein the centralized database can be accessed by multiple individuals via the mobile application or a web portal. In some aspects, the centralized database performs further analysis of the measurement data using additional complementary data. In some aspects, the centralized database automatically transmits an alert to a third party based on pre-defined parameters in response to the measurement data.

In any of the aspects, the analysis device presents a result and/or interpretive advice directly to an operator. In some aspects, the analysis device communicates wirelessly with a mobile phone or tablet for data presentation, interpretation, tracking and/or analysis.

### BRIEF DESCRIPTION OF THE DRAWINGS

For purposes of summarizing the disclosure, certain aspects, advantages, and novel features are discussed herein. It is to be understood that not necessarily all such aspects, advantages, or features will be embodied in any particular implementation of the disclosure, and an artisan would recognize from the disclosure herein a myriad of combinations of such aspects, advantages, or features.

FIG. 1: Structure of a conventional lateral flow assay.

FIG. 2A-2C: Representative structures of conventional electrochemical sensors with various electrode arrangements.

FIG. 3: Implementation of a lateral flow assay using ion selective electrodes and liposomes for detection of a target analyte via an analyte concentration-dependent signal.

FIG. 4: Implementation of a lateral flow assay using molecularly imprinted polymers for detection of a target analyte.

FIG. 5: Implementation of a lateral flow assay using aptamers for detection of a target analyte.

FIG. 6A: Implementation of a lateral flow assay where pH is the auxiliary measure, obtained using an integrated pH sensor.

FIG. 6B: The lateral flow assay of FIG. 6A is used to correct a measurement result.

FIG. 6C: The lateral flow assay of FIG. 6A is used to exclude a measurement result.

FIG. 6D: The lateral flow assay of FIG. 6A is used to improve clinical interpretation of a measurement result.

FIG. 7A: Implementation of a lateral flow assay where integrated microfluids serve to collect saliva directly from the tongue and then filter particulate matter and bubbles.

FIG. 7B: Implementation of a lateral flow assay where integrated microfluidics serve to collect saliva directly from the tongue and then automatically mix a running buffer with the collected sample.

FIG. 8A: Implementation of a lateral flow assay where a fluid detection electrode is positioned under the membrane to confirm progression of sample flow and used to initiate a measurement.

FIG. 8B: Implementation of a lateral flow assay where several membranes of variable flow speed are positioned to regulate the addition of a secondary compound required for initiation of a chemical reaction.

FIG. 9A: Implementation of lateral flow assay where a pre-metered liquid pack is manually opened by the system operator to initiate a step in the measurement process.

FIG. 9B: Implementation of lateral flow assay where a pre-metered liquid pack is opened by the analyzer device.

FIG. 10A: Implementation of a lateral flow assay where depression of a cutting element is used to isolate a region of the membrane.

FIG. 10B: Implementation of a lateral flow assay where depression of a cutting element both isolates a region of the membrane and causes the release of a liquid.

FIG. 11: Implementation of a lateral flow assay where a heating or cooling element is integrated to regulate the temperature of the temperature of the assay.

FIG. 12: Implementation of a lateral flow assay where a temperature sensing element is used as quality control element.

FIG. 13: Implementation of a lateral flow assay integrate electrode used for batch and/or type tracking.

### DETAILED DESCRIPTION OF THE PREFERRED EMBODIMENT

The present application describes various embodiments and features of a system and method for identification or quantification of analytes in biological samples. These analytes may be, but are not limited to, antibodies, proteins, peptides, hormones, nucleic acids, drugs, and pathogens. Although the following disclosure focuses on the analysis of saliva or blood, the implementations described below, or variations of those implementations, may be used for analysis of any other biological sample such as urine, fecal matter or sweat.

### Conventional Lateral Flow Assay Structure

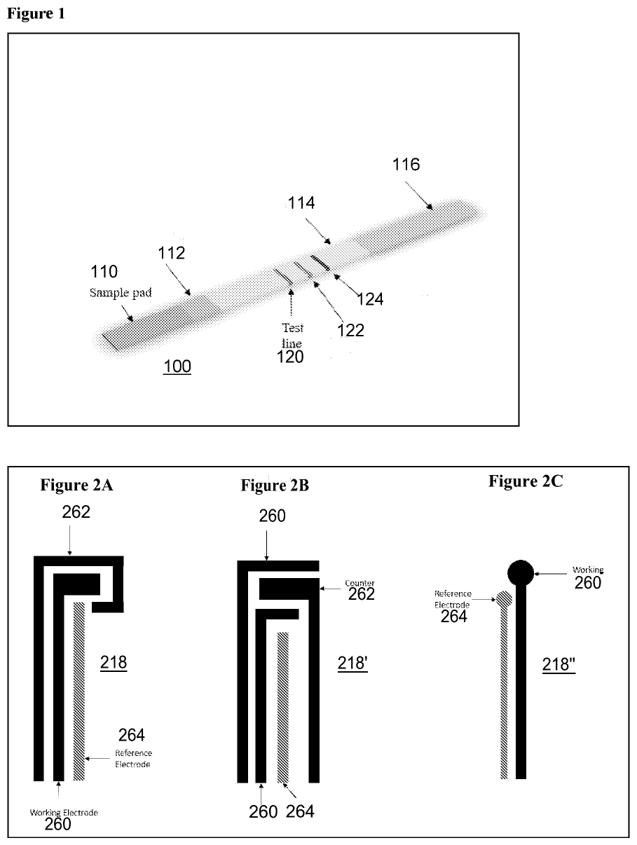

The structure of a conventional lateral flow assay (LFA) 100 is shown in FIG. 1.

In a conventional LFA 100, a biological sample is first added to a sample pad 110. In some implementations, the sample pad 110 includes cellulosic fiber filters or woven meshes. The sample pad 110 primarily acts to regulate the flow of sample into the downstream materials. The sample pad 110 may also act to modify sample parameters such as pH (for example, via the addition of reagents to the sample pad 110), act as a filter to remove particulate matter from the sample, or contain a blocking solution improve binding specificity.

In some implementations, sample parameters such as pH or viscosity may be normalized to facilitate a more sensitive measurement. Alternatively or additionally, the biological sample may be first mixed with a running buffer prior to placement on the sample pad 110. A similar approach involves the addition of running buffer to the sample pad 110 following the sample.

Next, the sample flows through a conjugate pad 112. In some implementations, conjugate pad 112 may include glass fiber filters, cellulose filters, surface treated polyester or polypropylene filters, or other filters. Conjugate pad 112 may contain one or several labelled molecules (the conjugate) capable of binding the target analyte(s) (for example, in the case of a “sandwich” LFA) or one or several labelled molecules analogous to the target analyte(s) (for example, in the case of a “competitive” LFA). The test sample rehydrates the conjugate in or on the conjugate pad 112 such that the conjugate is present in the sample at a consistent concentration.

Conjugate molecules include but are not limited to antigens, monoclonal or polyclonal antibodies, complementary nucleotide sequences or aptamers. Labels include but are not limited to enzymes (for example, horseradish peroxidase-HRP, alkaline phosphate-AP), electroactive components/redox mediators (for example, ferrocene, thionine's), metallic nanoparticles (for example, gold), polymeric microspheres (for example, latex), redox polymers with metal complexes, and liposomes. In some implementations, signaling tracers such as fluorescent, dye, enzymes, redox mediators and ions can be incorporated into liposomes. The LFA can therefore be implemented as an integrated sensor, for example an integrated immunosensor, integrated hydration sensor, integrated pH sensor, and the like.

The sample/conjugate mixture then flows along a membrane 114 upon which several capture reagents have been bound. In some implementations, membrane 114 can include one or more of nitrocellulose, cellulose acetate, glass fiber membranes, nylon, polyvinylidene, fluoride, and the like. A capture reagent can be an antigen, monoclonal or polyclonal antibodies, complementary nucleotide sequences, aptamer, or synthetic antibody such as molecular imprinted polymers.

At the position designated as the “test line” 120, a capture ligand is immobilized to capture the analyte or competitive conjugate molecule. A single LFA 100 may contain several test lines 120. In some implementations, an LFA 100 may include two, three, four, five, six, or more test lines 120, each with an immobilized capture ligand. In some implementations, two or more immobilized capture ligands are the same capture ligand. In some implementations, two or more immobilized capture ligands are different capture ligands.

At the position designated as the control line 122, a ligand is immobilized specific to a conjugate molecule, acting as a confirmation of conjugate solubilization and test validity. In some implementations, additional control lines 122 can include specific immobilized ligands selected to confirm additional sample features. One or more additional calibration lines 124 may be targeted at one or more unrelated targets, and may be included to control for any intrinsic background interference within the sample.

Lastly an absorbent pad or wick pad 116 is positioned after the test and control lines to encourage additional sample to flow across the membrane. In some implementations, wick pad 116 may be cellulose fiber filters, cotton fibers, cotton/glass blend, or other suitable materials. In some implementations, wick pad 116 may reduce background signal and increase test sensitivity.

### Lateral Flow Assay Formats

The two common assay formats used in lateral flow strips are sandwich and competitive assays.

Sandwich assays are used to detect relatively large analytes, such as proteins, whereas competitive assays are used for small analytes, such as hormones, where the analyte is too small for two capture molecules to bind simultaneously.

In a sandwich LFA, a capture molecule to one binding site on the analyte is conjugated to a label and a second capture molecule to another binding site is immobilized on the test line. The presence/concentration of the label on the test line is indicative of the presence/concentration of the target analyte.

In some implementations of a competitive LFA, a capture molecule targeted at a specific binding site is immobilized on the test line. A molecule containing this binding site is conjugated to a label, and then competes with any analyte present in the sample for binding on the test line. The presence/concentration of the label on the test line is indicative of the absence/inverse concentration of the target analyte.

In another implementation of a competitive LFA, the target analyte is immobilized on the test line. A labelled capture molecule binds to any analyte present in the sample and any remaining capture molecule will bind to the test line. Again, the presence/concentration of the label on the test line is indicative of the absence/inverse concentration of the target analyte.

### Lateral Flow Assay Detection Formats

In conventional LFAs the presence of the conjugate at the test or control line is determined with optical or electrochemical methods.

In case of optical measurements, the color intensity of the test lines is relative to conjugate concentration. For some labels, an additional reagent may need to be added to the LFA after the sample has run across the membrane to initiate a color-changing chemical reaction or enhance color intensity.

The measurement result may be determined by eye (typical for qualitative LFA tests). Alternatively, an external or integrated optical reader may be used to more objectively or quantitatively assess the intensity of the optical signal at the test and calibration lines.

In the case of electrochemical methods, one or several sensors are integrated within the lateral flow strip. The position of the sensors and the conjugate is chosen such that a signal is generated when the conjugate is proximal to the test or control line. For some conjugates an additional reagent may need to be added to the membrane to initiate an electrochemical measurement.

As illustrated in FIGS. 2A-2C, implementations of an electrochemical sensor 218 for electrochemical measurements may contain one or multiple working electrodes 260, counter electrodes 262, and reference electrodes 264. For example, electrochemical sensor 218 shown in FIG. 2A may include one working electrode 260, one counter electrode 262, and one reference electrode 264. Electrochemical sensor 218′ shown in FIG. 2B may include two working electrodes 260, one counter electrode 262, and one reference electrode 264. In some implementations, each working electrode 260 is paired to a respective counter electrode 262, and the strip includes a single reference electrode 264. In some implementations, counter electrode 262 is not necessary. For example, electrochemical sensor 218″ shown in FIG. 2C includes one working electrode 260 and one reference electrode 264, although in some implementations, multiple working electrodes 260 may be used with the one reference electrode 264. This type of electrochemical sensor may interface with an integrated or external analysis system, which may use one of various methods of measurement including amperometry, impedance, potentiometric, or voltammetry.

The bound conjugate generates a signal dependent on the relative concentration of the conjugate on the test or control line. This signal is proportional or inversely proportional to the analyte concentration.

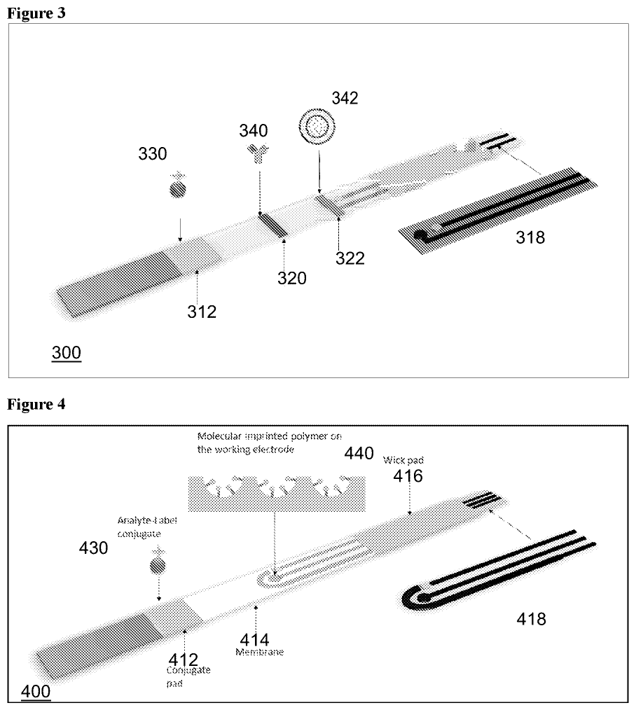

In some implementations, such as sensor 300 shown in FIG. 3, the sensor 300 may include an ion-selective electrode where the working electrode has an ion-selective membrane. In some implementations, sensor 300 may correspond to sensor 100 in some or all respects, including conjugate pad 312, test line 320, and control line 322, each of which generally correspond to respective conjugate pad 112, test line 120, and control line 122 as discussed above. In some implementations, sensor 300 can include an ion or ionic solution loaded liposome 342 immobilized above an ion selective electrode 318. A lysing agent may be conjugated to the target analyte as an analyte-lysis conjugate 330, which is loaded on the conjugate pad 312. In some implementations, an ion-loaded liposome may be tagged to a capture ligand.

When the conjugate molecule 330 is not captured by the capture ligand 340 (e.g., due to competition with analyte present in the sample), it continues to flow to the region containing the liposomes 342, disrupting the liposome bilayer, and generating an analyte dependent relative increase in ion concentration. The ion selective electrode 318 can be used to measure the ion concentration as an indicator of analyte concentration in the sample.

Molecularly imprinted polymers are an alternative to conventional antibody-based capture of analytes in lateral flow assays. In contrast to antibodies, these polymers are chemically inert, can withstand extreme pH and temperatures, have long term stability and are insoluble in water and most organic solvents.

In some implementations, such as sensor 400 shown in FIG. 4, the capture molecule is a molecular imprinted polymer 440 on the surface of the working electrode 418 as part of a competitive LFA. In some implementations, sensor 400 may correspond to sensor 100 in some or all respects, including conjugate pad 412, membrane 414, and wick pad 416, each of which generally correspond to respective conjugate pad 112, membrane 114, and wick pad 116 as discussed above. In some implementations of sensor 400, an analyte-label conjugate 430 is present in the conjugate pad 412 and competes with analyte present in the sample to bind to the polymer. When the labelled conjugate molecule 430 is captured by a molecularly imprinted polymer 440, a signal is generated which can act as an indicator of analyte concentration in the sample. The analyte concentration-dependent signal can be measured at electrode 418.

In some implementations, the label is conjugated to an antibody targeting the analyte as part of a sandwich LFA. Where the analyte is present, it is bound by the labelled antibody and captured by the polymer on the working electrode, generating a signal which can act as an indicator of analyte concentration in the sample.

Aptamers (single stranded nucleic acid molecules which bind with high affinity to a target molecule) can be used instead of or in combination with conventional antibody-based capture of analytes in lateral flow assays. In contrast to antibodies, aptamers have a higher thermal stability and can be mass manufactured through conventional polynucleotide manufacturing pathways.

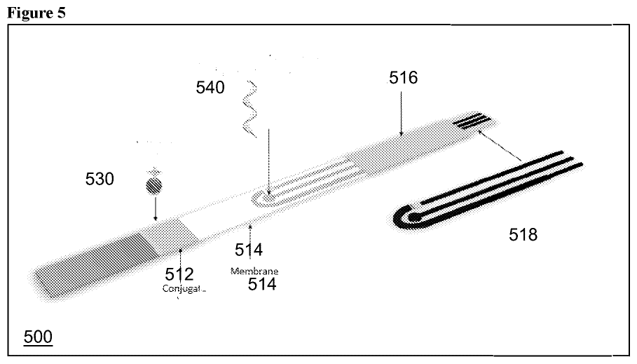

In some implementations such as senor 500 illustrated in FIG. 5, the capture molecule can be an aptamer 540 on the surface of the working electrode 418 as part of a competitive LFA. In some implementations, sensor 500 may correspond to sensor 100 in some or all respects, including conjugate pad 512, membrane 514, and wick pad 516, each of which generally correspond to respective conjugate pad 112 membrane 114, and wick pad 116 as discussed above. In some implementations of sensor 500, an analyte-label conjugate 530 is present in the conjugate pad 512 and competes with analyte present in the sample to bind to an aptamer 540. When the labelled conjugate molecule 530 is captured by an aptamer 540, a signal is generated which can act as an indicator of analyte concentration in the sample. Aptamer 540 may be immobilized on the working electrode 518, and/or the test line (not shown), and/or other appropriate location. The analyte concentration-dependent signal can be measured at electrode 518.

In some implementations, the label is conjugated to an antibody in an analyte-label conjugate 530 targeting the analyte as part of a sandwich LFA. Where the analyte is present, it is bound by the labelled antibody and captured by the aptamer 540 on the working electrode 518, generating a signal which can act as an indicator of analyte concentration in the sample.

### Use of Auxiliary Measurements to Correct, Exclude or Augment LFA Results

Many compounds and reactions typically used in an LFA are sensitive to reaction conditions such as temperature, pH, or ionic concentration. These sensitivities can be accounted for on the LFA to create a more robust integrated sensor, for example a robust integrated immunosensor.

One such compound is an enzyme, a biological molecule which increases the rate of a chemical reaction. Enzymes may be used within an LFA as the part of a conjugate molecule whereby the enzymatic reaction generates an optical or electrochemical signal. A specific enzyme will have an optimum pH for functioning, outside of which enzyme activity is reduced or ablated.

Another such compound is an aptamer, a single stranded nucleic acid molecule or short peptide which binds with high affinity to a target molecule. An aptamer's selectivity is sensitive to solution conditions (pH and ion concentration) which may impact the conformation of the target binding site, and subsequently performance, if not appropriately corrected.

As described above, these effects may be reduced using running buffers or chemical additives. In some contexts, this may not be possible or desirable due to the additional complexity introduced by these components or the additional diagnostic value of the modulating parameters.

In some implementations, an LFA is paired with one or multiple additional biosensors. These biosensors perform auxiliary measures to correct, exclude, augment, and/or aid in the interpretation of LFA measurements.

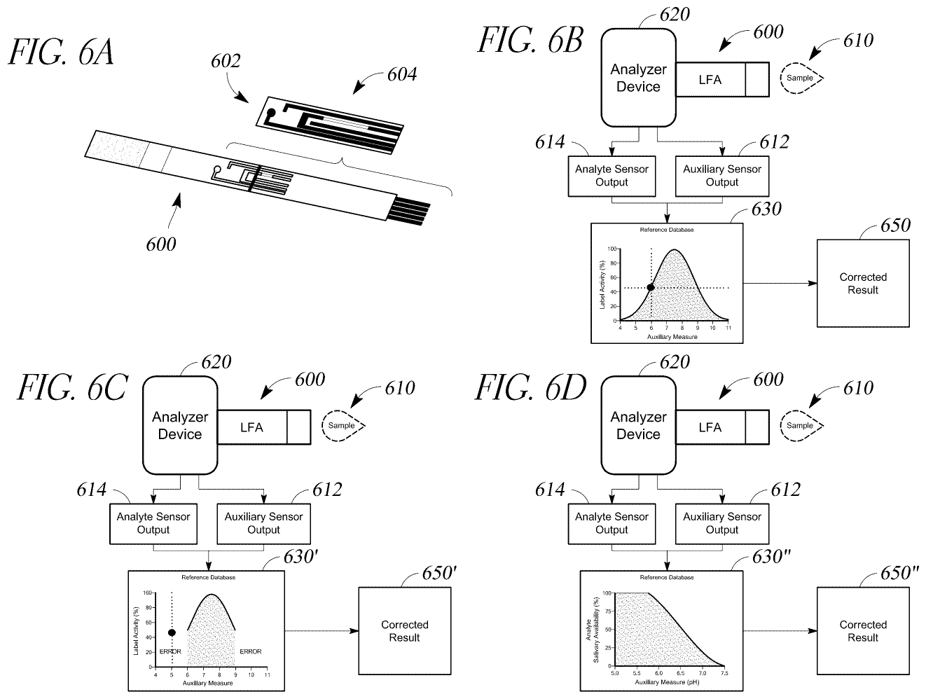

In some implementations, the auxiliary measure is pH, which may be determined using a pH sensor 602 integrated below the sample membrane of an analyte biosensor 604 on an LFA 600 as illustrated in FIG. 6A. This pH measurement may be used in one or more of multiple ways as shown in FIGS. 6B-6D.

In some implementations, for example as illustrated in FIG. 6B, pH may be known to affect the binding of conjugate to the test line or efficiency of an enzymatic reaction of a sample 610. LFA 600 and analyzer device 620 can be used to collect and measure parameters of sample 610. An analyte sensor output 614 from analyte sensor 604 is generated with an auxiliary sensor output 612 from auxiliary sensor (e.g., pH sensor) 602. A reference database 630 can be used with the measured pH 612 and measured analyte 612 for the correction of test strip output to produce a corrected result 650 to improve assay accuracy.

In some implementations, for example as illustrated in FIG. 6C, pH of a biological sample 610′ is within a range and samples outside of this range cannot be measured accurately. The pH can be used for identification of a sample outside of acceptable pH parameters to better identify an incorrect result. LFA 600 and analyzer device 620 can be used to collect and measure parameters of sample 610′. An analyte sensor output 614 from analyte sensor 604 is generated with an auxiliary sensor output 612 from auxiliary sensor (e.g., pH sensor) 602. A reference database 630′ can be used with the measured pH 612 and measured analyte 612 to exclude results of the test strip that are known to be outside the acceptable range, or to otherwise identify error our outlier data. The remaining results may be used produce an excluded result 650′ to improve assay accuracy.

In some implementations, for example as illustrated in FIG. 6D, pH of a biological sample 610″ is indicative of salivary flow rate, which impacts interpretation of analyte concentration due to biological processes. An enhanced pH assessment can provide additional diagnostics information, which may be used to better interpret the result of the LFA 600 LFA 600 and analyzer device 620 can be used to collect and measure parameters of sample 610″. An analyte sensor output 614 from analyte sensor 604 is generated with an auxiliary sensor output 612 from auxiliary sensor (e.g., pH sensor) 602. A reference database 630″ can be used with the measured pH 612 and measured analyte 612 to adjust or provide context for results of the test strip, and may be used produce an improved clinical interpretation result 650″ to improve assay accuracy.

In some implementations, pH may be measured and used one or more of the functions discussed above. For example, an LFA 600 and analyzer device 620 may be used to collect an analyte sensor output 614 and an auxiliary sensor output 612, where both outputs 612, 614 are used with multiple databases 630, 630′, and 630″ to provide a final result that is pH corrected, excludes erroneous or invalid data, and includes the improved clinical interpretation.

In some implementations, the auxiliary measure is osmolality or osmolarity and is determined using an impedimetric biosensor integrated below the sample membrane. This measurement may similarly be used to correct, exclude or augment LFA output.

In some implementations, the auxiliary measure is temperature and is determined using a temperature sensor integrated below the sample membrane and/or in the handheld measurement device. This measurement may similarly be used to correct, exclude or augment LFA output.

In some implementations, the auxiliary measurement is not integrated into the LFA but instead performed with an independent biosensor. In this implementation, an analysis device is capable of interfacing with two or more biosensors and performing a multi-component analysis integrating each of the measurements.

In some implementations, the auxiliary measurement is kinematic data and is collected with a second analysis device. In this implementation the second analysis device can comminate data with the first analysis device for multi-component analysis.

In some implementations, the auxiliary measurement is a set of answers and/or a set of observations that have been provided and/or annotated by the individual providing the sample or the individual operating the analyzer device. In this implementation the answers and/or observations are used to contextualize the LFA output.

In some implementations, a system of one or more analysis devices and one or more sensors cooperate to provide one or more assays corrected or interpreted with multiple auxiliary measurements. For example, in some implementations one analysis device can use one sensor that measures a bodily fluid analyte adjusted by auxiliary measurements of pH and temperature to provide a final measure of the analyte. In other example implementations, a system can include one analysis device with one sensor that measures a bodily fluid analyte adjusted by auxiliary measurements of pH and sample temperature, and a second analysis device with a first sensor for kinematic data and a second sensor for ambient temperature, where the system provides a final measure of the analyte adjusted for sample pH, sample temperature, kinematic data, and ambient temperature.

Integration of a Sample Collection and Processing Microfluidics within an LFA to Remove the Need for External Processing

For some conventional LFAs it is necessary to collect a sample with an external apparatus and/or perform some form of sample processing, such as addition of a running buffer, prior to application to the LFA sample pad.

These processes serve to normalize sample parameters such as pH, osmolarity or viscosity prior to measurement and/or to remove potential contaminants from the sample such as blood cells, bubbles, or food debris, which may interfere with downstream measurement processes. In some contexts, these processes are not desirable due to the additional complexity introduced.

In some implementations, an LFA is paired with integrated sampling microfluids to allow for direct sampling and/or automated processing of biological samples within an LFA.

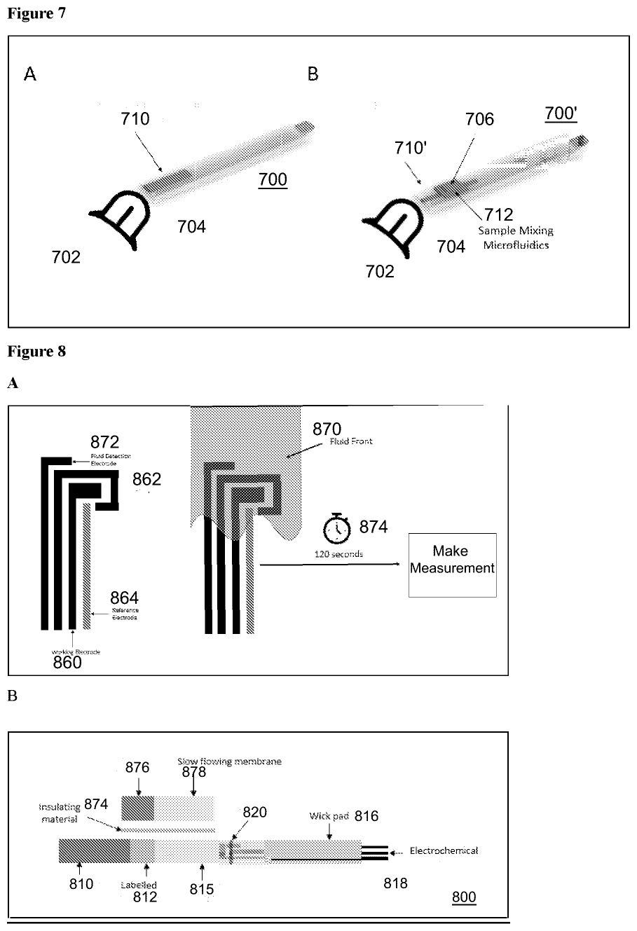

In some implementations, such as sensor 700 illustrated in FIG. 7A, integrated sample collection and processing microfluidics 710 serve to collect saliva 704 directly from the tongue 702 and then filter particulate matter and bubbles from the sample 704 prior to metering to the sample pad. In some implementations, such as sensor 700′ illustrated in FIG. 7B, integrated sample collection and processing microfluidics 710′ include the features of sample collection and processing microfluidics 710 discussed above, as well as sample mixing microfluidics 712. Sample collection and processing microfluidics 710′ serve to collect saliva 704 directly from the tongue 702 and then automatically mix a running buffer with the collected sample in sample mixing microfluidics 712, prior to metering to the sample pad. In some implementations, the buffer may be stored in a buffer reservoir 706 prior to mixing. In some implementations, mixing microfluidics 712 are separate from the sample collection and processing microfluidics 710.

In some implementations, the sample is collected in an external receptacle and then sampled with the LFA. In some implementations, integrated microfluidics serve to automatically mix a running buffer with the collected sample prior to metering to the sample pad.

In some implementations, the sample is collected in an external receptacle and then the receptable physically interfaces with the end of the LFA. The integrated microfluidics serve to automatically draw sample from the attached receptable and mix a running buffer with the collected sample prior to metering to the sample pad.

Integration of Automated Timing Elements within an LFA to Remove the Need for Manual Timing

For some conventional LFAs it necessary to manually perform additional steps following sample addition and prior to acquisition of a measurement result.

Examples of this include the addition of a buffer to initiate a chemical reaction, insertion of the LFA into a measurement device or folding of a secondary structure to change the direction of sample flow. Typically, these steps are prompted after a set time, or once a sample has progressed past a specific point on a membrane.

In some implementations, an LFA is paired with one or multiple integrated timing element which may be used to automate or facilitate a time-sensitive process.

Some implementations, such as electrochemical sensor 818 shown in FIG. 8A, can include this timing element. Electrochemical sensor 818 may correspond to sensor 218 in some or all respects, including working electrode 860, counter electrode 862, and reference electrode 864, each of which generally correspond to respective working electrode 260, counter electrode 262, and reference electrode 264 as discussed above. Electrochemical sensor 818 may also include a fluid detection electrode 872 positioned under the membrane to confirm progression of sample flow. For example, once fluid 870 has flowed a detection point or line on the membrane, the fluid detection electrode 872 may generate a signal used by the analyzer device to begin a timer 874. Timer 874 can be initiated in the analyzer device to count down for a predetermined time. The duration of timer 874 may be between 0.2 and 180 sec, for example, 0.2, 0.5, 1, 2, 5, 10, 15, 20, 30, 45, 60, 90, 120, 150, 180 s, or other durations within the range. In some implementations, the duration of timer 874 may be 0.5 to 10 minutes, for example, 0.5, 1, 1.5, 2, 3, 5, 8, or 10 minutes and other durations within the range. For example, a timer 874 for 2 minutes may be used count down to measurement initiation. This removes the need for a user to trigger the measurement once the reaction has sufficiently progressed. Other durations for timer 874 may be used as a pause or wait time for other functions, such as a delay to allow for adequate sample collection, to collect additional parameters as discussed above, to verify communications or other connectivity, etc.

In some implementations, a fluid detection electrode 872 is positioned near the wick pad 110 to indicate that the sample has flowed past the test line 120, control line 122, or calibration line 124. Detection of fluid at this point can be used to trigger the measurement. This removes the need for a user to monitor flow progress and trigger the measurement once sample has sufficiently progressed along the membrane 114.

In some implementations, several membranes of variable flow speed may be positioned to regulate the addition of a secondary compound required for initiation of a chemical reaction. For example, LFA 800 illustrated in FIG. 8B may include a test strip base that may correspond to LFA 100 in some or all respects, including sample pad 810, test line 820, wick pad 816, and electrode 818, each of which generally correspond to respective sample pad 110, test line 120, wick pad 116, and electrode 218 as discussed above. Sensor 800 may also include labeled conjugates 812, which correspond to conjugate pads 112, 312, 412, and 512 discussed above. Sensor 800 may also include a primary membrane 815 with a fast flow rate. A second membrane 878 may be chosen with a slower flow rate, such that delivery of the substrate solution 876 to the test line 820 is delayed relative to the primary membrane 815. This allows a first compound to effectively bind to the test line prior to delivery of the second compound. An insulating material 874 may be used to separate the fast-flowing primary membrane 815 from the slow-flowing secondary membrane 878.

In some implementations, several fluid detection electrodes are integrated into the LFA and unexpected output from the timing elements (e.g. more than 5 minutes pass between fluid detection at electrode one and fluid detection at electrode two) is used to flag a measurement error.

Integrated Packaging of Liquid Chemicals within a LFA to Reduce User Error when Adding Substrate Solutions

For some conventional LFAs it may not be possible to automate addition of chemicals prior to acquisition of a measurement result, such as where chemicals must be stored in a liquid phase and cannot be dried on the conjugate pad. In such cases, these chemicals may need to be stored separately from the LFA materials and manual addition of these chemicals by the operator may be required.

In some implementations, an LFA is paired with an integrated liquid pack which may be used to store a reagent. Opening of the pre-metered liquid pack results in the addition of a set volume of the reagent at a set location. The use of a liquid pack reduces the risk of user error by regulating the volume and location that liquid is deposited.

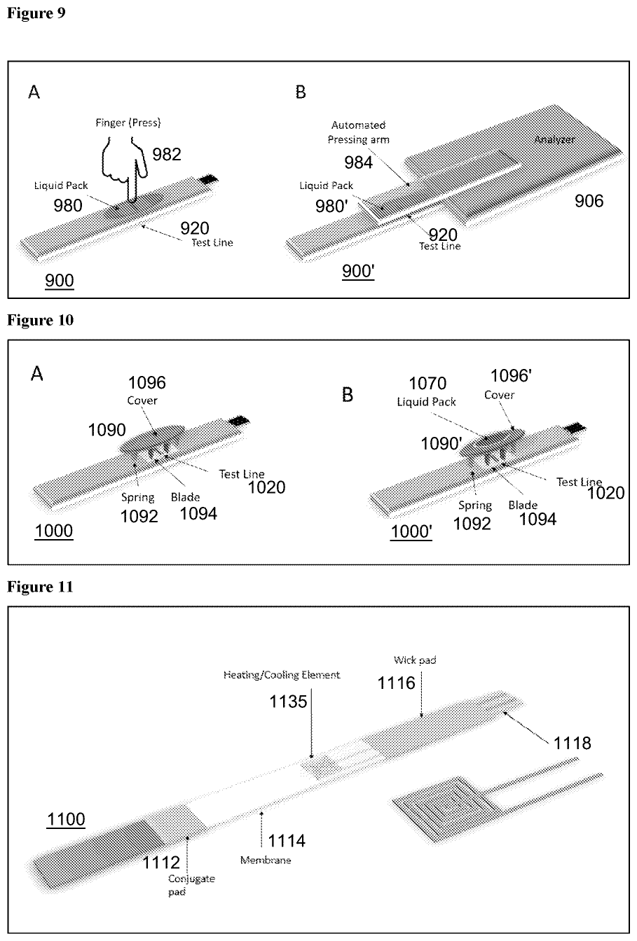

In some implementations, such as LFA 900 illustrated in FIG. 9A, an integrated liquid pack 980 is manually opened by the system operator 982 to initiate a step in the measurement process. Liquid pack 980 may be part of an enzyme substrate that is deposited onto the surface of the test line 920 following binding of an enzyme conjugate molecule.

In some implementations, such as LFA 900′ illustrated in FIG. 9B, the integrated liquid pack 980′ is opened by the analyzer device 986. For example, the analyzer device 986 may include an automated pressing arm 984 to open the liquid pack 980′ upon insertion of the LFA 900′ into the analyzer, or when the sample reaches test line 920. In some implementations, the analyzer device includes a piercing, cutting, or pinching mechanism to open the liquid pack 980′ when the LFA 900′ is inserted in the analyzer. Liquid pack 980′ and/or LFA 900′ may include a perforation, weakness, pin, rail, or other component to cooperate with the mechanism and/or analyzer for facilitating alignment and/or opening of the liquid pack 980′. These features may further reduce the likelihood of user error by regulating the timing and location in which the liquid pack 980′ is opened.

### Integration of a Cutting Element to Regulate Substrate Solution Deposition

In some LFAs, addition of substrate may be confounded due to the continued flow of solution into the absorbance pad, or reverse flow through to the sample and conjugate pad. To reduce the impact of this factor, it may be necessary to isolate the region of the membrane containing the test and control lines prior to the addition of a substrate solution.

In some implementations, an LFA is paired with an integrated cutting element which is used to isolate a segment of the lateral flow system.

In some implementations, such as LFA 1000 illustrated in FIG. 10A, depression of a cutting element 1090 results in cutting of the membrane with one or more blades 1094. The blades 1094 may be positioned at or near test line 1020, and arranged such that they do not sever the electrodes required for a measurement. In some implementations, cutting element 1090 may include a cover 1096 or button for ease of operation. Cover 1096 may also help evenly distribute pressure over multiple blades 1094. In some implementations, cutting element 1090 may also include a spring 1092, arranged to hold the cutting element open and return the blades to a raised position when pressure is released.

In some implementations, such as LFA 1000′ illustrated in FIG. 10B, the integrated cutting element 1090′ may be combined with an integrated liquid pack 1070. Cutting element 1090′ corresponds to cutting element 1090 in many respects, including cover 1096, spring 1092, and blade 1094 as discussed above. As cutting element 1090′ is pressed, the liquid pack 1070 is also pressed to release the liquid. The same pressing motion causes the cutting element 1090′ to lower blades 1094 to isolate the region of the membrane where the substrate solution is being released.

### Integration of a Heating or Cooling Element to Improve LFA Performance

For some LFA, performance such as sample flow, analyte binding, signal generation etc., may be impacted by the temperature of the LFA and/or sample. In some situations, it may be difficult, or may not be possible, to compensate for these effects using a temperature sensor, and instead it is necessary or more convenient to modify the temperature of the LFA.

In some implementations, an LFA incorporates a temperature modifier, such as a heating and/or cooling element which is used to modify the temperature of the LFA to a preferred temperature.

In some implementations, such as LFA 1100 illustrated in FIG. 11, the temperature modifier 1135 may be a heating element implemented, for example, as an integrated resistive heating coil. LFA 1100 may be similar to the other LFA devices described above, including conjugate pad 1112, membrane 1114, and wick pad 1116, which each respectively correspond to conjugate pad 112, membrane 114, and wick pad 116 discussed above. When the LFA 1100 is inserted into the analysis device, the temperature of the LFA 1100 is determined using an integrated temperature sensing element. If the temperature is too low, the analysis system powers the temperature modifier 1135 (heating element) to heat the device until the desired temperature is achieved, as determined by the temperature sensing element, and then prompts the user to apply the sample for analysis.

In some implementations, the temperature modifier 1135 may be a cooling element implemented, for example, as an integrated fluid pack containing two separated chemicals (e.g., urea and water). When the LFA, for example LFA 1100, is inserted into the analysis device, the temperature of the LFA 1100 is determined using an integrated temperature sensing element. If the temperature is too high, the analysis system activates the temperature modifier 1135 to cool the device. In some implementations, the temperature modifier 1135 mixes the two fluids resulting in endothermic reaction which cools the LFA 1100. When the desired temperature is achieved as determined by the temperature sensing element and then prompts the user to apply the sample for analysis. In some implementations, the temperature modifier 1135 heats or cools the LFA as discussed above. In some implementations, the temperature modifier 1135 both heats and cools as determined by the temperature sensor. In addition to those discussed above, other temperature modifying mechanisms, such as air or liquid cooling, can be used as appropriate with a pump, fan, or the like. In some implementations, time is used to adjust the temperature, for example by waiting a predetermined time for a sample to warm up or cool down.

In some implementations, the heating or cooling element 1135 is integrated into the analysis system and the LFA, for example LFA 1100, contains an integrated material with high thermal conductivity. When the LFA 1100 is inserted into the analysis device, the temperature of the LFA 1100 is determined using an integrated temperature sensing element (not shown). Where the temperature is outside of the desired range, the temperature modifier 1135 activates heating or cooling elements to modify the temperature within the analysis system, which in turn modifies the temperature of the LFA 1100 via the conductive element. When the desired temperature is achieved, as determined by the temperature sensing element, the analysis system then prompts the user to apply the sample for analysis.

### Integration of a Quality Control Element to Prevent Inappropriate LFA Use

For some LFA there may be chemical or material components which are sensitive to appropriate storage conditions such as temperature or humidity and/or have a limited shelf life. Furthermore, for some LFA (such as those which use electrochemical rather than optical methods) it may not be apparent whether the LFA has previously been used.

In some implementations, an LFA incorporates a quality control element which can be used to identify whether a test strip has been stored appropriate, has expired, or has been previously used.

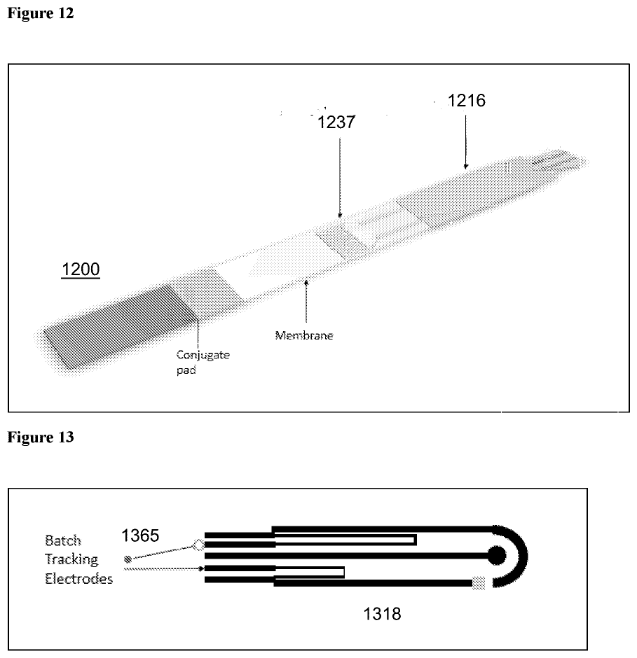

In some implementations, such as LFA 1200 illustrated in FIG. 12, the quality control element 1237 is a temperature sensing element integrated in the LFA 1200. LFA 1200 may be similar to the other LFA devices described above, including conjugate pad 1212, membrane 1214, and wick pad 1216, which each respectively correspond to conjugate pad 112, membrane 114, and wick pad 116 discussed above. Where the LFA 1200 has been exposed to a temperature above a threshold value, the properties (e.g., color, resistance, phase) of the quality control element 1237, (e.g., temperature sensing element) are irreversible modified. When the LFA 1200 is inserted into the analysis device, the properties of the temperature sensing element 1237 are measured by the device. Where the properties indicate exposure to a temperature above the threshold value, the LFA 1200 is identified as expired and cannot be used to perform an analysis.

In some implementations, the quality control element 1237 is a moisture detecting element integrated in the LFA, for example LFA 1200 within the wick pad 1216. Where the LFA 1200 has been previously used to analyze a sample, the quality control element 1237 (e.g., moisture detecting element) will have been wetted and the properties of the quality control element 1237 are irreversibly modified. When the LFA 1200 is inserted into the analysis device, the properties of the moisture detecting element 1237 are measured by the device. Where the properties indicate exposure to moisture, the LFA 1200 is marked as used and cannot be used to perform an analysis.

In some implementations, an LFA 1200 with a quality control element 1237 will be identified as expired, as discussed above. In some implementations, an expired LFA may marked as such. For example, expired LFAs 1200 can be visually marked with a stamp, hole, brand, or other visible indicator, or electrically marked by severing a trace, shorting a connection, etc. Integration of a batch tracking element to assist with test strip identification.

For analysis systems which are compatible with multiple types of LFA it is necessary to include a mechanism which allows for the system to identify the type of LFA, initiate the correct measurement process, and appropriately interpret the LFA output.

Furthermore, due to manufacturer and batch-to-batch variation in LFA manufacturing it may also be necessary to interpret LFA output using manufacturer-specific or batch-specific reference or calibration data. To accomplish this, a mechanism is needed which can identify the LFA manufacturer and/or batch. In the present invention an LFA incorporates an electrode, or other structure, which can be used to identify LFA type, manufacturer, and/or batch.

In some implementations, such as circuitry 1318 illustrated in FIG. 13, the electrode arrangement 1318 for a specific LFA production batch is positioned and sized as to have a specific resistance. In this sense, the particular resistance operates as an identifier for an LFA manufacturer, batch, and/or type. After the LFA carrying the electrode arrangement or circuitry 1318 is inserted into the analysis device, this resistance is measured by the analysis device and used to identify the LFA type, manufacturer, and/or batch. The analysis device refers to a reference database to identify the correct measurement process and/or calibration data associated with this LFA type, manufacturer, and batch.

In some implementations, encoded batch and type tracking data may additionally be used to identify if a test strip is out of date, counterfeit, region-locked, proprietary, and the like. In some implementations, resistance-based identification on circuitry 1318 can be used to trigger a firmware update of the analysis unit to enable appropriate processing of the LFA carrying the circuitry 1318. For example, batch encoding 1318 in an electrode arrangement 1318 may prompt a handheld device to download additional software or firmware to enable measuring of an additional analyte or to update a reference database (for example, reference database 630 with correction or calibration parameters as discussed above) for appropriate processing.

Communication of Data with a Central Databased to Facilitate Analysis and Data Use

In some implementations, the analyzer device presents a result and/or interpretive advice directly to an operator. For example, results, including raw, corrected, calibrated, adjusted, excluded, and interpreted results may be presented to a user of the system, such as an individual providing a biofluid sample, a person operating the analysis device, or a remote user. Data may be presented with a user interface, for example to select, mark, adjust, zoom, collate, or otherwise view, change, manipulate, and/or annotate the data. To improve the utility of measurement data, in some implementations it may be beneficial for LFA results to be transmitted to a central database. These results may then be relayed to other individuals for action or as part of a more complex analysis.

In some implementations, the analysis system communicates with a mobile device to relay LFA results to a centralized database. In some implementations, the analyzer device alternatively or additionally communicates wirelessly with a mobile phone or tablet for data presentation, interpretation, tracking and/or analysis.

In some implementations, the analysis system wirelessly communicates the LFA result, and accompanying information, to an internet connected mobile device, which subsequently relays the result to the centralized database. This database may be accessed via the individual providing the sample, the individual operating the analysis system, or other individuals authorized to view the measurement result. In some implementations, the centralized database may be accessed via a mobile application and/or web portal.

In some implementations additional analysis of the LFA result is performed on the centralized database, optionally integrating the result and other data, to help interpret the LFA results.

In some implementations the centralized database automatically alerts a third party based on the measurement result if meets one or more specific criteria, to help improve the immediacy of response to a LFA result.

### Other Variations

Although some implementations describe LFA devices, the systems, devices, and/or methods disclosed herein can be applied to other types of therapies usable standalone or in addition to LFA diagnostics. Systems, devices, and/or methods disclosed herein can be extended to any medical device, and in particular any diagnostic device testing bodily fluids, though other fluid testing may also be appropriate. For example, systems, devices, and/or methods disclosed herein can be used with devices that provide one or more of additional diagnostics and/or treatment based on the diagnostics. The systems and methods disclosed herein are not limited to medical devices and can be utilized by any liquid testing device.

Any implementations of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, implementation, or example are to be understood to be applicable to any other aspect, implementation or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing implementations. The protection extends to any novel one, or any novel combination, of the features disclosed in this specification (including any accompanying claims, abstract and drawings), or to any novel one, or any novel combination, of the steps of any method or process so disclosed.

While certain implementations have been described, these implementations have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some implementations, the actual steps taken in the processes illustrated and/or disclosed may differ from those shown in the figures. Depending on the implementation, certain of the steps described above may be removed, others may be added. For example, the actual steps and/or order of steps taken in the disclosed processes may differ from those shown in the figure. Depending on the implementation, certain of the steps may be implemented in different locations or structures. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific implementations disclosed above may be combined in different ways to form additional implementations, all of which fall within the scope of the present disclosure.

User displays and interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, and/or displayed in any suitable order.

Provided herein are lateral flow assay devices and methods of using such devices to detect biomarkers, analytes, ionic concentrations, and the like in fluid samples from a subject. One of skill in the art will understand that such lateral flow assay devices may be used to detect any of the biomarkers described herein or additional biomarkers.

The term “immobilized” or “embedded” interchangeably refers to reversibly or irreversibly immobilized molecules (e.g., analytes or binding agents). In some examples, reversibly immobilized molecules are immobilized in a manner that allows the molecules, or a portion thereof (e.g., at least about 25%, 50%, 60%, 75%, 80% or more of the molecules), to be removed from their immobilized location without substantial denaturation or aggregation. For example, a molecule can be reversibly immobilized in or on an absorbent material (e.g., an absorbent pad) by contacting a solution containing the molecule with the absorbent material, thereby soaking up the solution and reversibly immobilizing the molecule. The reversibly immobilized molecule can then be removed by wicking the solution from the absorbent material, or from one region of the absorbent material to another. In some cases, a molecule can be reversibly immobilized on an absorbent material by contacting a solution containing the molecule with the absorbent material, thereby soaking up the solution, and then drying the solution-containing absorbent material. The reversibly immobilized molecule can then be removed by contacting the absorbent material with another solution of the same or a different composition, thereby solubilizing the reversibly immobilized molecule, and then wicking the solution from the absorbent material, or from one region of the absorbent material to another.

Irreversibly immobilized molecules (e.g., binding agents or analytes) are immobilized such that they are not removed, or not substantially removed, from their location under mild conditions (e.g., pH between about 4-9, temperature of between about 4-65° C.). Exemplary irreversibly immobilized molecules include those described above, in addition to protein analytes or binding agents bound to a nitrocellulose, polyvinylidene fluoride, nylon or polysulfide membrane by standard blotting techniques (e.g., electroblotting). Other exemplary irreversibly immobilized molecules include protein analytes or binding agents bound to glass or plastic (e.g., a microarray, a microfluidic chip, a glass histology slide or a plastic microtiter plate having wells with bound protein analytes therein).

The term “binding agent” refers to an agent that specifically binds to a molecule such as an analyte. While binding agents, aptamers, ions, binding molecules, and analytes are described in many contexts herein, it will be understood by one of skill in the art that other binding agents can be used instead, as preferred by the user. A wide variety of binding agents are known in the art, including antibodies, aptamers, affixers, lipocalins (e.g., anticalins), thioredoxin A, bilin binding protein, or proteins containing an ankyrin repeat, the Z domain of staphylococcal protein A, or a fibronectin type III domain. Other binding agents include, but are not limited to, biotin/streptavidin, chelating agents, chromatography resins, affinity tags, or functionalized beads, nanoparticles and magnetic particles.

The terms “bind” and “effectively bind” refer to a molecule (e.g., binding agent such as an aptamer) that binds to a target with at least 2-fold greater affinity than non-target compounds, e.g., at least about 4-fold, 5-fold, 6-fold, 7-fold, 8-fold, 9-fold, 10-fold, 20-fold, 25-fold, 50-fold, 100-fold, 1000-fold, or more than 1000-fold greater affinity.

Conditional language used herein, such as, among others, “can,” “could”, “might,” “may,” “e.g.,” and the like, unless specifically stated otherwise, or otherwise understood within the context as used, is generally intended to convey that certain implementations include, while other implementations do not include, certain features, elements and/or states. Thus, such conditional language is not generally intended to imply that features, elements and/or states are in any way required for one or more implementations or that one or more implementations necessarily include logic for deciding, with or without author input or prompting, whether these features, elements and/or states are included or are to be performed in any particular implementations. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain implementations require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain implementations, examples and applications, it will be understood by those skilled in the art that the present disclosure extends beyond the specifically disclosed implementations to other alternative implementations and/or uses and obvious modifications and equivalents thereof, including implementations which do not provide all of the features and advantages set forth herein. Accordingly, the scope of the present disclosure is not intended to be limited by the specific disclosures of preferred implementations or embodiments herein, and may be defined by claims as presented herein or as presented in the future.

We claim:

1. A system for quantifying the concentration of one or more analytes in a biological sample using a lateral flow system with:
a first sensor which generates a signal relative to the concentration of the one or more analytes;
one or more additional sensors for one or more respective auxiliary measures to assist in interpretation of the signal of the first sensor; and
an analysis device which facilitates testing.

2. The system of claim 1 wherein at least one of the one or more additional sensors is integrated into the lateral flow system.

3. The system of any of claims 1-2 wherein at least one of the one or more additional sensors uses the same analysis device but is not integrated into the lateral flow system.

4. The system of any of claims 1-3 wherein at least one of the one or more auxiliary measures is pH.

5. The system of any of claims 1-4 wherein at least one of the one or more auxiliary measures is the concentration of an ion.

6. The system of any of claims 1-5 wherein at least one of the one or more auxiliary measures is osmolarity.

7. The system of any of claims 1-6 wherein at least one of the one or more auxiliary measures is temperature.

8. The system of any of claims 1-7 wherein at least one of the one or more auxiliary measures is used to correct the signal of the first sensor.

9. The system of any of claims 1-8 wherein at least one of the one or more auxiliary measures is used to flag an error.

10. The system of any of claims 1-9 wherein at least one of the one or more auxiliary measures is used to provide diagnostic context for the signal of the first sensor.

11. The system of any of claims 1-10 wherein at least one of the one or more auxiliary measures is data collected with a secondary analysis device.

12. The system of any of claims 1-11 wherein at least one of the one or more auxiliary measures is a set of answers provided by an individual providing the biological sample.

13. The system of any of claims 1-12 wherein at least one of the one or more auxiliary measures is a set of observations annotated by an individual operating the system.

14. The system of any of claims 1-13 wherein at least one of the one or more auxiliary measures is used to provide diagnostic context for the signal of the first sensor.

15. The system of any of claims 1-14 wherein the analysis device presents a result and/or interpretive advice directly to an operator.

16. The system of any of claims 1-15 wherein the analysis device communicates wirelessly with a mobile phone or tablet for data presentation, interpretation, tracking and/or analysis.

17. A system for quantifying the concentration of one or more analytes in a biological sample using a lateral flow system with:
one or more sensors which each generate a signal relative to the concentration of a respective one of the one or more analytes;
integrated microfluidics and materials for collection and processing of the biological sample; and
an analyzer device which facilitates testing.

18. The system of claim 17 wherein the integrated microfluidics and materials control for physical variability of the biological sample.

19. The system of claim 18 wherein the physical variability comprises viscosity or bubble content.

20. The system of any of claims 17-19 wherein the integrated microfluidics and materials control for chemical variability of the biological sample.

21. The system of claim 20 wherein the chemical variability comprises pH or ionic content.

22. The system of any of claims 17-21 wherein the analyzer device presents a result and/or interpretive advice directly to an operator.

23. The system of any of claims 17-22 wherein the analyzer device communicates wirelessly with a mobile phone or tablet for data presentation, interpretation, tracking and/or analysis.

24. A system for quantifying the concentration of one or more analytes in a biological sample using a lateral flow system with:
one or more sensors which each generate a signal relative to the concentration of a respective one of the one or more analytes;
one or more integrated automatic timing elements; and
an analyzer device which facilitates testing.

25. The system of claim 24 wherein the one or more timing elements comprises multiple membranes of variable flow speed.

26. The system of any of claims 24-25 wherein the one or more timing elements comprises a fluid detection electrode which initiates a timer on the analyzer device.

27. The system of any of claims 24-26 wherein the one or more timing elements comprises a fluid detection electrode which initiates a measurement.

28. The system of any of claims 24-27 wherein the one or more timing element comprises a fluid detection electrode which is positioned to identify that a sufficient sample has been collected.

29. The system of any of claims 24-28 wherein an output of the one or more timing elements is used to flag an error.

30. The system of any of claims 24-29 wherein the analyzer device presents a result and/or interpretive advice directly to an operator.

31. The system of any of claims 24-30 wherein the analyzer device communicates wirelessly with a mobile phone or tablet for data presentation, interpretation, tracking and/or analysis.

32. A system for quantifying the concentration of one or more analytes in a biological sample using a lateral flow system with:
one or more sensors which each generates a signal relative to the concentration of a respective one of the one or more analytes;
an integrated packaging of liquid required to initiate a chemical reaction; and
an analyzer device which facilitates testing.

33. The system of claim 32 wherein the integrated packaging of liquid is manually opened by a system operator to initiate a step in a measurement process.

34. The system of any of claims 32-33 wherein the integrated packaging of liquid is automatically opened by the analyzer device to initiate a step in a measurement process.

35. The system of any of claims 32-34 wherein the analyzer device presents a result and/or interpretive advice directly to an operator.

36. The system of any of claims 32-35 wherein the analyzer device communicates wirelessly with a mobile phone or tablet for data presentation, interpretation, tracking and/or analysis.

37. A system for quantifying the concentration of one or more analytes in a biological sample using a lateral flow system with:
one or more sensors which each generates a signal relative to the concentration of a respective one of the one or more analytes;
an integrated cutting element to isolate a segment of the lateral flow system; and
an analyzer device which facilitates testing.

38. The system of claim 37 wherein the cutting element is combined with an integrated liquid pack.

39. The system of any of claims 37-38 wherein the analyzer device presents a result and/or interpretive advice directly to an operator.

40. The system of any of claims 37-39 wherein the analyzer device communicates wirelessly with a mobile phone or tablet for data presentation, interpretation, tracking and/or analysis.

41. A system for quantifying the concentration of one or more analytes in a biological sample using a lateral flow system with:
one or more integrated ion selective electrodes;
ion loaded liposomes;
a conjugate molecule which acts to lyse the liposomes; and
an analyzer device which facilitates testing.

42. The system of claim 41 wherein the ion loaded liposomes are immobilized close to the one or more ion selective electrodes.

43. The system of claim 42 wherein the presence of a target analyte in the biological sample prevents capture of the conjugate molecule by a capture ligand, leading to lysis of the liposome and an increase in ion concentration.

44. The system of any of claims 41-43 wherein the analyzer device presents a result and/or interpretive advice directly to an operator.

45. The system of any of claims 41-44 wherein the analyzer device communicates wirelessly with a mobile phone or tablet for data presentation, interpretation, tracking and/or analysis.

46. A system for quantifying the concentration of one or more analytes in a biological sample using a lateral flow system with:
one or more integrated electrodes;
one or more molecularly imprinted polymers targeted at the one or more analytes;
one or more labeled conjugate molecules; and
an analyzer device which facilitates testing.

47. The system of claim 46 wherein each of the one or more molecularly imprinted polymers is positioned on a respective one of the one or more integrated electrodes.

48. The system of any of claims 46-47 wherein at least one of the one or more labeled conjugate molecules compete with at least one of the one or more analytes to bind to at least one of the one or more molecularly imprinted polymers.

49. The system of any of claims 46-48 wherein at least one of the one or more labeled conjugate molecules binds to at least one of the one or more analytes and the resulting complex binds to at least one of the one or more molecularly imprinted polymers.

50. The system of any of claims 46-49 wherein the analyzer device presents a result and/or interpretive advice directly to an operator.

51. The system of any of claims 46-50 wherein the analyzer device communicates wirelessly with a mobile phone or tablet for data presentation, interpretation, tracking and/or analysis.

52. A system for quantifying the concentration of one or more analytes in a biological sample using a lateral flow system with:
one or more integrated electrodes;
one or more aptamers targeted at the one or more analytes;
one or more labeled conjugate molecules; and
an analyzer device which facilitates testing.

53. The system of claim 52 wherein the one or more aptamers is each positioned on a respective one of the one or more integrated electrodes.

54. The system of any of claims 52-53 wherein at least one of the one or more labeled conjugate molecules competes with at least one of the one or more analytes to bind to at least one of the one or more aptamer.

55. The system of any of claims 52-54 wherein at least one of the one or more labeled conjugate molecules binds to at least one of the one or more analytes and the resulting complex binds to at least one of the one or more aptamers.

56. The system of any of claims 52-55 wherein the analyzer device presents a result and/or interpretive advice directly to an operator.

57. The system of any of claims 52-56 wherein the analyzer device communicates wirelessly with a mobile phone or tablet for data presentation, interpretation, tracking and/or analysis.

58. A system for quantifying the concentration of one or more analytes in a biological sample using a lateral flow system with:
one or more integrated electrodes;
one or more temperature modifying elements; and
an analyzer device which facilitates testing.

59. The system of claim 58 wherein the one or more temperature modifying elements comprises a resistive heating element integrated into the lateral flow system.

60. The system of any of claims 58-49 wherein the one or more temperature modifying elements comprises a liquid pack which when opened results in an endothermic reaction, lowering the temperature of the lateral flow system.

61. The system of any of claims 58-60 wherein at least one of the one or more temperature modifying elements is located within the analyzer device and the temperature of the lateral flow system is modified using a thermally conductive material.

62. The system of any of claims 58-61 wherein the analyzer device prompts a user to only apply a sample for analysis once a desired temperature has been reached.

63. A system for quantifying the concentration of one or more analytes in a biological sample using a lateral flow system with:
one or more integrated electrodes;
one or more integrated quality control elements; and
an analyzer device which facilitates testing.

64. The system of claim 63 wherein at least one of the one or more quality control elements is irreversibly modified by exposure to a temperature outside of a desired range.

65. The system of any of claims 63-64 wherein at least one of the one or more quality control elements is irreversibly modified by exposure to a humidity outside of a desired range.

66. The system of any of claims 63-65 wherein at least one of the one or more quality control elements is irreversibly modified once the lateral flow system has been used.

67. The system of any of claims 63-66 wherein the analyzer device automatically measures at least one of the one or more quality control elements and does not perform analysis when the at least one quality control element indicates inappropriate storage or use.

68. A system for quantifying the concentration of one or more analytes in a biological sample using a lateral flow system with:
one or more integrated electrodes for type and/or batch tracking;
an analyzer device which facilitates testing; and
a mobile application.

69. The system of claim 68 wherein type and/or batch tracking is accomplished using the resistance value of an integrated electrode.

70. The system of any of claims 68-69 wherein type and/or batch tracking is used to indicate whether a lateral flow test is out of date.

71. A system for quantifying the concentration of one or more analytes in a biological sample using a lateral flow system with:
one or multiple integrated electrodes;
an analyzer device which facilitates testing; and
a mobile application.

72. The system of claim 71 wherein the analyzer device wirelessly communicates measurement data with an internet connected mobile device.

73. The system of any of claims 71-72, further comprising a centralized database for receiving measurement data from the analyzer device, wherein the centralized database can be accessed by multiple individuals via the mobile application or a web portal.

74. The system of claim 73 wherein the centralized database performs further analysis of the measurement data using additional complementary data.

75. The system of claim 73 wherein the centralized database automatically transmits an alert to a third party based on pre-defined parameters in response to the measurement data.
